(19) United States
(12) Patent Application Publication
(10) Pub. No.: US 2007/0141083 A1
(43) Pub. Date: Jun. 21, 2007

(54) Use of a single-cell protein material

(76) Inventors: Rolf Berge, Bones (NO); Gunnar Kleppe, Hafrsjord (NO)

Correspondence Address: REED SMITH LLP, 3110 FAIRVIEW PARK DRIVE, FALLS CHURCH, VA, 22042, US

(21) Appl. No.: 10/563,273

(22) PCT Filed: Jul. 2, 2004
(86) PCT No.: PCT/NO04/00204

§ 371 Date: Jul. 24, 2006

(30) Foreign Application Priority Data
Jul. 4, 2003 (NO) 20033082

(51) Int. Cl. A61K 39/02

(52) U.S. Cl. 424/234.1

(57) ABSTRACT

The present invention relates to the use of a single-cell protein material (SCP). The SCP material lowers the concentration of cholesterol in plasma, and triglycerides in the liver. SCP also induces a favourable change in the fatty acid pattern, and lowers the concentration of homocysteine in plasma. A preferable embodiment of the invention relates to the use of SCP as an anti-atherogenic and cardio protective agent, either given as a pharmaceutical or as nutritional composition, e.g. as a functional food.

### FIELD OF INVENTION

The present invention relates to the use of a single-cell protein material (SCP). The SCP material lowers the concentration of cholesterol in plasma, and triglycerides in the liver. SCP also induces a favourable change in the fatty acid pattern, and lowers the concentration of homocysteine in plasma. A preferable embodiment of the invention relates to the use of SCP as an anti-atherogenic and cardio protective agent, either given as a pharmaceutical or as a functional food. Further, the invention relates to the use of the SCP material as a nutritional composition.

### BACKGROUND OF THE INVENTION

Recently, much attention has been directed toward the development of new sources of protein which may be incorporated into foods for human and/or animal consumption. A number of different protein-containing materials have been proposed as substitutes for more traditional sources of protein, such as fish meal, soy products and blood plasma, in human foods and as animal feeds. These materials include single-cell microorganisms such as fungi, yeasts and bacteria which contain high proportions of proteins. These may be grown by single-cell reproduction, and several bio-synthetic processes for the production of protein through the growth of single-cell microorganisms on hydrocarbon or other substrates have been developed. Today, the most widely used protein-containing microorganisms (also referred to herein as “single-cell proteins”) are those derived from fungi or yeast. Single-cell protein materials can be used directly in foods, e.g. as a spray dried product.

WO 01/60974 discloses a process for imparting functional properties to a single-cell material. The claimed product can be used as a gelling agent or emulsifier.

The present inventors have shown that a single-cell protein material in accordance with the invention has several beneficial biological effects, and that such a material can be used as a pharmaceutical or as a functional food.

We have shown that the single-cell protein material lowers the concentration of plasma cholesterol and homocysteine, and also lowers the concentration of hepatic triacylglycerols. Based on these findings, it is anticipated that the single-cell material will have a preventive and/or therapeutic effect on stenosis, atherosclerosis, coronary heart disease, thrombosis, myocardial infarction, stroke and fatty liver. Treatment with a single-cell protein material represents a new way to treat these diseases.

Unicellular organisms such as bacteria consist of a large number of extremely small cells each containing protein encapsulated within a cell-wall structure.

Conveniently, the single-cell material may be produced by a fermentation process in which oxygen and a suitable substrate such as a liquid or gaseous hydrocarbon, an alcohol or carbohydrate, e.g. methane, methanol or natural gas, together with a nutrient mineral solution are fed to a tubular reactor containing the microorganisms. A number of such processes are well known and described in the art.

Particularly preferred for use in the invention are single-cell protein materials derived from fermentation on hydrocarbon fractions or on natural gas. Especially preferred are single-cell proteins derived from the fermentation of natural gas. As the concentration of microorganisms increases within the fermentor, a portion of the reactor contents or broth is withdrawn and the microorganisms may be separated by techniques well known in the art, e.g. centrifugation and/or ultrafiltration. Conveniently, in such a fermentation process, the broth will be continuously withdrawn from the fermentor and will have a cell concentration between 1% and 5% by weight, e.g. about 3% by weight.

Single-cell materials produced from two or more microorganisms may be used in accordance with the invention. Although these may be produced in the same or separate fermentors, generally these will be produced in the same fermentor under identical fermentation conditions. Materials produced from separate fermentation processes may be blended together prior to homogenization.

Preferred bacteria for use in the invention include Methylococcus capsulatus (Bath), a thermophilic bacterium originally isolated from the hot springs in Bath, England available from Norferm Danmark AS, Odense, Denmark. M. capsulatus (Bath) has optimum growth at about 45° C., although growth can occur between 37° C. and 52° C. It is a gram-negative, non-motile spherical cell, usually occurring in pairs. The intracellular membranes are arranged as bundles of vesicular discs characteristic of Type I methanotrophs. M capsulatus (Bath) is genetically a very stable organism without known plasmids. It can utilize methane or methanol for growth and ammonia, nitrate or molecular nitrogen as a source of nitrogen for protein synthesis.

Other bacteria suitable for use in the invention include the heterotrophic bacteria Alcaligenes acidovorans DB3, Bacillus firmus DB5 and Bacillus brevis DB4 which each have optimum growth at a temperature of about 45° C. These strains are available from Norferm Danmark AS, Odense, Denmark.

A. acidovorans DB3 is a gram-negative, aerobic, motile rod belonging to the family Pseudomonadaceae which can use ethanol, acetate, propionate and butyrate for growth. B. brevis DB4 is a gram-negative, endospore-forming, aerobic rod belonging to the genus Bacillus which can utilize acetate, D-fructose, D-mannose, ribose and D-tagatose. B. firmus DB5 is a gram-negative, endospore-forming, motile, aerobic rod of the genus Bacillus which can utilize acetate, N-acetyl-glucosamine, citrate, gluconate, D-glucose, glycerol and mannitol.

Suitable yeasts for use in the process of the invention may be selected from the group consisting of Saccharomyces and Candida. One example of a fermentation process which uses natural gas as the sole carbon and energy source is that described in EP-A-306466 (Dansk Bioprotein). This process is based on the continuous fermentation of the methanotropic bacteria M. capsulatus grown on methane. Air or pure oxygen is used for oxygenation and ammonia is used as the nitrogen source. In addition to these substrates, the bacterial culture will typically require water, phosphate (e.g. as phosphoric acid) and several minerals which may include magnesium, calcium, potassium, iron, copper, zinc, manganese, nickel, cobalt and molybdenum, typically used as sulphates, chlorides or nitrates. All minerals used in the production of the single-cell material should be of food-grade quality.

Natural gas mainly consists of methane, although its composition will vary for different gas fields. Typically, natural gas may be expected to contain about 90% methane, about 5% ethane, about 2% propane and some higher hydrocarbons. During the fermentation of natural gas, methane is oxidized by methanotrophic bacteria to biomass and carbon dioxide. Methanol, formaldehyde and formic acid are metabolic intermediates. Formaldehyde and to some extefit carbon dioxide are assimilated into biomass. However, methanotrophic bacteria are unable to use substrates comprising carbon-carbon bonds for growth and the remaining components of natural gas, i.e. ethane, propane and to some extent higher hydrocarbons, are oxidized by methanotrophic bacteria to produce the corresponding carboxylic acids (e.g. ethane is oxidized to acetic acid). Such products can be inhibitory to methanotrophic bacteria and it is therefore important that their concentrations remain low, preferably below 50 mg/l, during the production of the biomass. One solution to this problem is the combined use of one or more heterotrophic bacteria which are able to utilize the metabolites produced by the methanotrophic bacteria.

Such bacteria are also capable of utilizing organic material released to the fermentation broth by cell lysis. This is important in order to avoid foam formation and also serves to minimize the risk of the culture being contaminated with undesirable bacteria. A combination of methanotrophic and heterotrophic bacteria results in a stable and high yielding culture.

During production of the single-cell material, the pH of the fermentation mixture will generally be regulated to between about 6 and 7, e.g. to 6.5±0.3. Suitable acids/bases for pH regulation may be readily selected by those skilled in the art. Particularly suitable for use in this regard are sodium hydroxide and sulphuric acid. During fermentation the temperature within the fermentor should preferably be maintained to within the range of from 40° C. to 50° C., most preferably 45° C.±2° C.

Especially preferred for use in the invention is a microbial culture comprising a combination of the methanotrophic bacterium Methylococcus capsulatus (Bath), and the heterotrophic bacteria Alcaligenes acidovorans DB3 and Bacillus firmus DB 5 optionally in combination with Bacillus brevis DB4 (all strain available from Norferm Danmark AS, Odense, Denmark). The role of A. acidovorans DB3 is to utilize acetate and propionate produced by M. capsulatus (Bath) from ethane and propane in the natural gas. A. acidovorans DB3 may account for up to 10%, e.g. about 6 to 8%, of the total cell count of the resulting biomass. The role of B. brevis DB4 and B. firmus DB5 is to utilize lysis products and metabolites in the medium. Typically, B. brevis DB4 and B. fermis DB5 will each account for less than 1% of the cell count during continuous fermentation.

Suitable fermentors for use in preparing the single-cell material are those of the loop-type, such as those described in DK 1404/92, EP-A-418187 and EP-A-306466 of Dansk Bioprotein, or air-lift reactors. A preferable reactor is described in applicant's PCT application WO 03/016460, which is incorporated herein by reference. A loop-type fermentor having static mixers results in a high utilization of the gases (e.g. up to 95%) due to the plug-flow characteristics of the fermentor. Gases are introduced at several positions along the loop and remain in contact with the liquid until they are separated into the head space at the end of the loop. Continuous fermentation may be achieved using 2-3% biomass (on a dry weight basis) and a dilution rate of 0.02 to 0.50 h⁻¹, e.g. 0.05-0.25 h⁻¹.

Other fermentors may be used in preparing the single-cell material and these include tubular and stirred tank fermentors.

Ideally, the biomass or single-cell material produced from fermentation of natural gas will comprise from 60 to 80% by weight crude protein; from 5 to 20% by weight crude fat; from 3 to 10% by weight ash; from 3 to 15% by weight nucleic acids (RNA and DNA); from 10 to 30 g/kg phosphorus; up to 350 mg/kg iron; and up to 120 mg/kg copper. Particularly preferably, the biomass will comprise from 68 to 73%, e.g. about 70% by weight crude protein; from 9 to 11%, e.g. about 10% by weight crude fat; from 5 to 10%, e.g. about 7% by weight ash; from 8 to 12%, e.g. about 10% by weight nucleic acids (RNA and DNA); from 10 to 25 g/kg phosphorus; up to 310 mg/kg iron; and up to 110 mg/kg copper. Preferable, the amino acid profile of the protein content should be nutritionally favourable with a high proportion of the more important amino acids cysteine, methionine, threonine, lysine, tryptophan and arginine. Typically these may be present in amounts of about 0.7%, 3.1%, 5.2%, 7.2%, 2.5% and 6.9%, respectively (expressed as a per cent of the total amount of amino acids). Generally the fatty acids will comprise mainly the saturated palmitic acid (approx. 50%) and the monounsaturated palmitoleic acid (approx. 36%). The mineral content of the product will typically comprise high amounts of phosphorus (about 1.5% by weight), potassium (about 0.8% by weight) and magnesium (about 0.2% by weight).

Generally, single-cell protein materials obtained from a continuous fermentation process will be subjected to centrifugation and filtration, e.g. ultrafiltration, processes to remove most of the water present and to form an aqueous paste or slurry. During centrifugation the dry matter content of the biomass is typically increased from about 2 to about 15% by weight, e.g. to about 12% by weight. Ultrafiltration, which may be effected at a temperature of between 40 and 50° C., e.g. between 42 and 46° C., further concentrates the biomass to a product containing from 10 to 30%, preferably from 15 to 25%, e.g. from 15 to 22% by weight single-cell material. The size exclusion used during ultrafiltration will generally be in the range of about 100,000 Daltons.

Following ultrafiltration the biomass may be cooled, preferably to a temperature of from 10 to 30° C., e.g. to about 15° C., for example by passing the concentrated protein slurry from the ultrafiltration unit over a heat exchanger after which it may be held in a buffertank at constant temperature, e.g. for a period of from 1 to 24 hours, preferably 5 to 15 hours, e.g. 5 to 12 hours, at a temperature of from 10 to 20° C., more preferably from 5 to 15° C. at a pH in the range of from 5.5 to 6.5. Optionally, the single-cell protein material may be sterilized.

Further, the single-cell material may optionally be homogenized in order to distrupt the cell wall structure. The homogenization can be conducted in any conventional way, but may be carried out in a conventional high pressure homogenizer in which the cells may be ruptured by first pressurizing, e.g. up to a pressure of 150 MPa (1500 bars), and then depressurizing the inside of the homogenizer. Preferably, the total pressure drop applied to the biomass will be in the range of from 40 MPa to 120 MPa (400 to 1200 bar), e.g. about 80 MPa (800 bar). The drop in pressure may be stepped, i.e. this may comprise one or more steps, although generally this will comprise one or two steps, preferably a single step. In cases where homogenization is effected as a two-step process it is preferable that the pressure drop in the second step should represent less than ⅕, preferably less than 1/10, e.g. about 1/20 of the total pressure drop in the homogenizer. The temperature of the material during homogenization should preferably not exceed 50° C. The homogenization step is detailed in applicant's PCT application WO01/60974, which is incorporated herein by reference.

The homogenization process herein described results in the production of a product comprising, preferably consisting essentially of ruptured cell material. For example, ruptured cell material will be present in an amount of at least 80%, preferably at least 90% by weight. Typically, the product will be a relatively viscous protein slurry containing soluble and particulate cellular components. Although this may be used directly as an additive in food products or as pharmaceuticals, this will usually be further processed whereby to remove excess water from the product. The choice of any additional drying step or steps will depend on the water content of the product following homogenization and the desired moisture content of the final product.

Typically, the product will be further processed in accordance with spray drying techniques well known in the art. Any conventional spray drier with or without fluid bed units may be used, for example the Type 3-SPD spray drier available from APV Anhydro, Denmark. Preferably the inlet temperature for the air in the spray drier may be about 300° C. and the outlet temperature may be about 90° C. Preferably the resulting product will have a water content of from about 2 to 10% by weight, e.g. from 6 to 8% by weight. The resulting product will typically be of a particle size of from 0.1 to 0.5 mm.

Particularly preferably, the step of homogenization will be immediately followed by spray drying. Alternatively, it may be necessary, or indeed desirable, to store or hold the homogenized product, e.g. in a storage or buffer tank, prior to further processing. In such cases, it has been found that the conditions under which the product is stored may reduce the gelling properties of the final product following spray drying. The gelling properties of the homogenized material may be maintained by storing this at a temperature of less than 20° C. and at a pH<7, preferably <6.5, particularly preferably at a pH in the range 5.5 to 6.5, e.g. 5.8 to 6.5. Under these conditions, the product may be stored for up to 24 hours without any substantial loss of gelling properties.

We have shown that the single-cell material has several beneficial biological effects, e.g. the ability to lower the concentration of cholesterol in plasma and liver. The materials also increases the mitochondrial β-oxidation. The single-cell material can thus in accordance with the pressent invention be used as a pharmaceutical composition.

Further, the single-cell material is especially useful as a functional component in food products, particularly when used as a substitute for natural plasma in animal feeds and in pet foods. When used in pet foods, additional ingredients may be added to the product such as fats, sugars, salt, flavourings, minerals, etc. The product may then be formed into chunks resembling natural meat chunks in appearance and texture. The product of the invention has the further advantages that this is readily formulated to contain necessary nutrients, is easily digested by the animals and is palatable to the animals.

### DETAILED DESCRIPTION OF THE INVENTION

The present invention relates to a single-cell protein material for the preparation of a pharmaceutical or nutritional preparation for the treatment and/or prevention of atherosclerosis, coronary heart disease, stenosis, thrombosis, myocardial infarction, stroke and fatty liver.

The experimental data clearly show that the SCP material according to the invention lowers the concentration of homocysteine in plasma. Homocysteine is a risk factor in diseases such as atherosclerosis, coronary heart disease, stenosis, thrombosis, myocardial infarction and stroke, and it is thus anticipated that the SCP material of the invention will be effective in preventing and treating these diseases.

The data also show that the level of triacylglycerols in the liver is decreased by administration of SCP, and it is anticipated that the SCP material can be used for the treatment and prevention of fatty liver.

A further embodiment of the present invention relates to a single-cell protein material for the preparation of a pharmaceutical or nutritional composition for the treatment and/or prevention of hypercholesterolemia, as we have shown that said material is capable of lowering the plasma concentration of cholesterol.

A still further embodiment relates to the use of a single-cell protein material for the preparation of a pharmaceutical or nutritional composition for lowering the concentration of homocysteine in the plasma. A hyperhomocysteine level can be established before the above indicated diseases are manifested. The administration of the SCP material has a general homocysteine lowering effect, and material of the present invention is thus especially suited for preventing the onset of, and lowering the risk for the above indicated diseases.

The results further indicate that the SCP material has general cardio and artery protective features, and we anticipate that the material can be given to decrease the risk for artery and cardio related diseases.

An object of the present invention is to administer the material either as a prophylactic or pharmaceutical drug, or as a functional feed or food material. The material can be given to human and non-human animals.

A preferred embodiment of the invention relates to a feed material comprising the single-cell protein material. The material can for instance be used for feeding agricultural animal, such as gallinaceous birds, bovine, ovine, caprine or porcine mammals, domestic or pet animal, such as dog or cat, and fish or shellfish, such as salmon, cod, Tilapia, clams, oysters, lobster or crabs.

A preferred embodiment of the invention uses SCP material produced by the fermentation of a microbial culture comprising methanotrophic bacteria. A more preferred embodiment fiter comprises one or more species of heterotrophic bacteria. A preferred embodiment uses a combination of the methanotrophic bacterium Methylococcus capsulatus (Bath), and the heterotrophic bacteria Alcaligenes acidovorans DB3 and Bacillus firmus DB 5, optionally in combination with Bacillus brevis DB4 (all strain available from Norferm Danmark AS, Odense, Denmark).

### FIGURE LEGENDS

FIG. 1 shows that the single-cell material (SCP) decreases the concentration of cholesterol in plasma.

FIG. 2 shows that the single-cell material (SCP) decreases the concentration of triacylglycerols in the liver.

FIG. 3 shows that the SCP material inhibits the ACAT enzyme.

FIG. 4 shows that the single-cell material increases the mitochondrial β-oxidation.

### DEFINITIONS USED IN THE APPLICATION

Animals

In this context the term “animals” include humans and farm (agricultural) animals, especially the animals of economic importance such as, bovine, ovine, caprine and porcine mammals, especially those that produce products suitable for the human consumption, such as meat, eggs and milk. Further, the term is intended to include fish and shellfish, such as salmon, cod, Tilapia, clams and oysters. The term also includes domestic animals such as dogs and cats.

Treatment

In relation to the pharmaceutical applications of the invention the term “treatment” refers to a reduction of the severity of the disease.

Prevention

The term “prevention” refers to the preventing of a given disease, i.e. a compound of the present invention is administered prior to the onset of the condition. This means that the compounds of the present invention can be used as prophylactic agents or as ingredients in functional foods or feed in order to prevent the risk or onset of a given disease.

Single-cell protein material (SCM is a material comprising single-cell microorganisms. The microorganisms can inter alia be fungi, yeasts and bacteria. The SCP material preferable contains high proportions of proteins.

### ADMINISTRATION OF THE COMPOUNDS OF THE PRESENT INVENTION

Preferable, the material according to the invention is administered orally, although any known kind of administration route or regime can be used. For oral pharmacological compositions such carrier material as, for example, water, gelatine, gums, lactose, starches, magnesium-stearate, talc, oils, polyalkene glycol, petroleum jelly and the like may be used. Such pharmaceutical preparation may be in unit dosage form and may additionally contain other therapeutically valuable substances or conventional pharmaceutical adjuvants such as preservatives, stabilising agents, emulsifiers, buffers and the like. The pharmaceutical preparations may be in conventional liquid forms such as tablets, capsules, dragees, ampoules and the like, in conventional dosage forms, such as dry ampulles, and as suppositories and the like.

In addition, the compounds of the present invention are appropriately administered in combination with other treatments for combatting or preventing a specific disease.

The invention will be more fully understood by reference to the following examples. They should not, however, be construed as limiting the scope of the invention.

As a nutritional composition the single-cell material may be formulated in any conventional way to a feed or food product.

### EXPERIMENTAL SECTION

The following non-limiting examples serve to further illustrate the invention.

Chemicals

[1-¹⁴C] palmitoyl-L-carnitine (54 Ci/mmol) was purchased from Amersham. The chemicals used for real-time RT-PCR was from Applied Biosystems. All other chemicals were obtained from common commercial sources and were of reagent grade.

Single-Cell Protein (SCP) Material

The SCP material used in the experiments was produced as given in example 1.

Animals and Treatments

4-5 weeks old male obese Zucker rats (Crl:(ZUC)/faBR) from Charles River, Germany, averaging 120±3 g at the start of the experiment, were kept in a room maintained at 12 hours light-dark cycles, at a temperature of 20±3° C., and relative humidity of 65±15%. The day after arrival the rats were randomised and placed separately in metabolic cages and divided into three experimental groups, each of six animals. The rats were adapted to the experimental conditions and experimental diets for 4 days, after which the faeces were collected for 7 days. The semipurified diets (Table 1), contained 20% crude protein (N×6,25) in the form of SCP or casein (control).

TABLE 1
Composition of the experimental diets

| g/kg diet | SCP | Casein |
|---|---|---|
| Protein | 270 | 217.6 |
| Soybean oil ¹ | 100 | 100 |
| Sucrose | 110 | 110 |
| Vitamins ² | 10 | 10 |
| Minerals ³ | 30 | 30 |
| Cellulose | 20 | 20 |
| NaCl | — | 21.8 |
| Dextrin | 496.1 | 490.6 |

¹ Fatty acid composition of the soybean oil (g/100 g fat): 18:2n − 6 (54.1 ± 0.5), 18:1n − 9 (21.8 ± 0.2), 16:0 (11.2 ± 0.1), 18:3n − 3 (6.1 ± 0.2), 18:0 (3.7 ± 0.1), 18:1n − 7 (1.5 ± 0.1), 20:0 (0.5 ± 0.1), 22:0 (0.5 ± 0.1).
² Vitamins (mg/kg diet): 8 mg vit. A (4000 I.U.), 2 mg vit. D3 (1000 I.U.), 60 mg vit. E (30 I.U.), 0.1 mg vit. K (0.05 I.U.), 1000 mg choline hydrogentartrate, 4 mg thiamine, 3 mg riboflavin, 6 mg pyridoxine, 20 mg niacin, 8 mg Ca-pantothenat, 1 mg folin, 5 mg vit. B12 (0.05 I.U.).
³ Minerals (g/kg diet): 8.5 g CaCO₃, 6.2 g CaHPO₄ × 2H₂O, 12.3 g KH₂PO₄, 1.4 g MgCO₃, 0.4 NaCO₃, 0.8 g NaCl, 0.02 g CuSO₄ × 5H₂O, 0.002 g NaF, 0.0002 g KI, 0.2 g FeSO₄ × H₂O, 0.05 g ZnSO₄ × H₂O.

The animals were daily offered equal feed rations, which were adjusted to meet the demand of the growing animal. The animals had free access to tap water. The rats were fed for 22 or 23 days after acclimatisation (three rats from each group were killed on day 22 and the rest on day 23), and the body weight was measured weekly. At the end of the feeding period, the animals were anaesthetised subcutaneously by 1:1 Hypnorm®/Dormicum® (Fentany/fluanisone-Midazolam), 0.2 mL/100 g body weight. Cardiac puncture was performed to collect blood samples (in heparin), and the liver was dissected. Parts of the liver were immediately frozen in liquid N₂, while the rest of the liver was chilled on ice for homogenisation. The protocol was approved by the Norwegian State Board of Biological Experiments with Living Animals.

Preparation of Subcellular Fractions

Livers from the rats were homogenised individually in ice-cold sucrose-solution (0.25 mol/L sucrose in 10 mmol/L-HEPES buffer pH 7.4 and 1 mmol/L EDTA) using a Potter-Elvehjem homogeniser. The subcellular fractions were isolated as described in Berge, R. K. et al (Berge, R. K., Flatmark, T. & Osmundsen, H. (1984), Enhancement of long-chain acyl-CoA hydrolase activity in peroxisomes and mitochondria of rat liver by peroxisomal proliferators. Eur J BiocheM 141: 637-644). Briefly, the homogenate was centrifuged at 1000×g for 10 min to separate the post-nuclear from the nuclear fraction. A mitochondrial-enriched fraction was prepared from the post-nuclear fraction at 10000×g for 10 min. A peroxisome-enriched fraction was prepared by centrifugation of the post-mitochondrial fraction at 23 500×g for 30 min. A microsomal-enriched fraction was isolated from the post-peroxisomal fraction at 100 000×g for 75 min. The remaining supernatant was collected as the cytosolic fraction. The procedure was performed at 0-4° C., and the fractions were stored at −80° C. Protein was assayed using the BioRad protein assay kit (BioRad, Heraules, Calif.) and bovine serum albumin as standard.

Enzyme Assays

Carnitine palmitoyltransferase I (CPT-I) activity was measured essentially as described by Bremer (Bremer, J. (1981), The effect of fasting on the activity of liver carnitine palmitoyltransferase and its inhibition by malonyl-CoA. Biochim Biophys Acta 665: 628-631). The assay for CPT-I contained 20 mmol/L HEPES pH 7.5, 70 mmol/L KCl, 5 mmol/L KCN, 100 μmol/L palmitoyl-CoA, 10 mg BSA/mL, and 0.6 mg tissue protein/mL. The reaction was started with 200 μmol/L [methyl-¹⁴C] L-carnitine (200 cpm/nmol). Assay conditions for CPT-II were identical except that BSA was omitted and 0.01% Triton X-100 was included. Tissue protein concentration was 2.5 μg/mL. Acyl-coenzyme A cholesterol acyltransferase (ACAT) was measured by using 130 mg protein and ¹⁴C-oleyl-CoA as substrate. The product was separated on TLC plates using hexane:diethylether:acetic acid (80;20:1) as the mobile phase, and counted in a scintillation counter (Win Spectral 1414 liquid scintillation counter, Wallac). 3-Hydroxy-3-methylglutaryl (HMG)-CoA reductase was measured by using 80 mg protein and ¹⁴C-HMG-CoA as a substrate. The product was separated on TLC plates using acetone:benzene (1:1) as the mobile phase, and counted in a scintillation counter. Fatty acid synthase was measured as described by Roncari (Roncari, D. A. (1981) Fatty acid synthase from human liver. Methods Enzymol 71 Pt C: 73-79), modified according to Skorve et al. (Skorve, J., al-Shurbaji, A., Asiedu, D., Bjorkhem, I., Berglund, L. & Berge, R. K. (1993) On the mechanism of the hypolipidemic effect of sulfur-substituted hexadecanedioic acid (3-thiadicarboxylic acid) in normolipidemic rats. J Lipid Res 34: 1177-1185), and acetyl-CoA carboxylase was determined by measuring the amount of NaH¹⁴CO₃ incorporated into malonyl-CoA.

Lipid Analysis

Lipids in whole liver and heparinised plasma were measured in the Tecnicon Axon system (Miles, Tarrytown, N.Y.), with the Bayer triglyceride and cholesterol enzymatic kits (Bayer, Terrytown, N.Y.) and the PAP 150 phospholipid enzymatic kit (bioMérieux, Lyon, France). Liver lipids were first extracted according to Bligh and Dyer (Bligh, E. G. & Dyer, W. J. (1959) A rapid method of total lipid extraction and purification. Can J Biochem Physiol 37: 911-91).

Faecal Sterols

Faecal total bile acids were prepared according to Suckling et al. (Suckling, K. E., Benson, G. M., Bond, B., Gee, A., Glen, A., Haynes, C. & Jackson, B. (1991), Cholesterol lowering and bile acid excretion in the hamster with cholestyramine treatment, Atherosclerosis 89: 183-190) with some modifications. Two mL of NaBH in ethanol (mg/mL) was added to 0.1 g of powdered dry feces. The mixture was allowed to react for 1 hour at ambient temperature, after which 50 μl of 2 mol/L HCl was added to remove any excess of NaBH. Neutral sterols were extracted from the samples with n-hexan (two consecutive washings) before the samples were hydrolysed over night with 200 μl 10 mol/L NaOH at 110° C. 240 μl of the hydrolysate together with 2.8 mL water was applied to Bond Elut C¹⁸ columns (Varian, 200 mg, 3 mL), that had previously been activated by 3 mL methanol and 3 mL water. Bile acids were retained in the columns, which were washed twice with 3 mL of 20% methanol in water, before the bile acids were eluted with 3 mL of methanol. The bile acids were air-dried at 45° C. and resolved in 1 mL of isopropanol. Total bile acids were determined enzymatically using a total bile acid diagnostic kit (Sigma 450A) on the Tecnicon Axon system.

Amino Acids

Amino acids in the diets were determined after hydrolysis in 6 M HCl at 110±2° C. for 22 hours and pre-derivatisation with phenylisothiocyanate according to the method of Cohen and Strydom (34). Total cysteine in the feeds was determined after oxidation of cysteine and cystine with 9:1 performic acid (88%): H₂O₂ (30%) (v/v) to yield cysteic acid. The samples were then hydrolysed in 6 M HCl at 110±2° C. for 22 hours and further treated as the amino acid analysis described above. Amino acids in liver and plasma were determined in a Biochrom 20 plus Amino Acid Analyzer (Amersham Pharmacia Biotech, Sweden) equipped with a lithium column with post column ninhydrin derivatization as previously described (24). Prior to analysis, liver samples were extracted and deproteinated by the addition of 2 volumes of 5% sulfosalisylic acid, kept on ice for 30 min and centrifuged at 5000×g for 15 min. The supernatants were mixed 4:1 (v/v) with internal standard (2.5 mmol/L Norleucine in 0.1 mol/L HCl). Plasma samples were mixed 1:1 with internal standard (1 mmol/L Norleucine in 0.1 mol/L HCl), centrifuged at 10000×g for 5 min before the supernatant was centrifuged in a filter tube (cut off 10 kDa, Biomax PB polyethersulfbne membrane, Millipore Corp., USA) at 10000×g for 30 min.

Fatty Acid Composition

Fatty acids were extracted from the samples with 2:1 chloroform: methanol (v/v) (35). The samples were filtered, saponified and esterified in 12% BF₃ in methanol (v/v). Fatty acid composition of total lipids from liver and plasma was analysed using methods described by Lie and Lambertsen (Lie, O. & Lambertsen, G. (1991) Fatty acid composition of glycerophospholipids in seven tissues of cod (Gadus morhua), determined by combined high-performance liquid chromatography and gas chromatography. J Chromatogr 565: 119-129). Fatty acid methyl esters were separated using a Carlo Erba gas chromatograph (‘cold on column’ injection, 69° C. for 20 s, increase at 25° C. min⁻¹ to 160° C. and hold at 160° C. for 28 min, increase at 25° C. min⁻¹ to 190° C. and hold at 190° C. for 17 min, increase at 25° C. min⁻¹ to 220° C. and hold at 220° C. for 9 min) equipped with a 50 m CP-sil 88 (Chrompack, Middelburg, The Netherlands) fused silica capillary column (i.d. 0.32 mm). The fatty acids were identified by retention time using standard mixtures of methyl esters (Nu-Chek-Prep, Elyian, Minn., N, USA). The fatty acid composition (weight percentage) was calculated using an integrator (Turbochrom Navigator, Version 4.0) connected to the GLC.

Lipids were extracted from plasma triacylglycerol-rich lipoprotein fraction using a mixture of chloroform and methanol, and separated by thin layer chromatography on silica gel plates (0.25 mm Silica gel 60, Merck) developed in hexane-diethyl ether-acetic acid (80:20:1, v/v/v) and visualized using Rhodamine 6G (0.05% in methanol, Sigma) and UV light. The spots were scraped off and transferred to tubes containing heneicosanoic acid (21:0) as internal standard. BF₃-methanol was added to the samples for transesterification. To remove neutral sterols and non-saponifiable material, extracts of fatty acyl methyl esters were heated in 0.5 mol/L KOH in ethanol-water solution (9:1). Recovered fatty acids were then re-esterified using BF₃-methanol. The methyl esters were analyzed on a GC8000Top gas chromatograph (Carlo Erba Instrument), equipped with a flame ionization detector (FID), programmable temperature of vaporization injector, AS 800 autosampler (Carlo Erba Instrument) and a capillary column (60 m×0.25 mm) containing a highly polar SP 2340 phase with film thickness 0.20 μm (Supelco). The initial temperature was 130° C., heating 1.4° C./min to final temperature 214° C. The injector temperature was 235° C. The detector temperature was 235° C., using hydrogen (25 mL/min), air (350 mL/min) and nitrogen as make-up gas (30 mL/min). The samples were run with constant flow using hydrogen as a carrier gas (1.6 mL/min). The splitting ratio was 20:1. The methyl esters were positively identified by comparison to known standards (Larodan Fine Chemicals, Malmo, Sweden) and verified by mass spectrometry. Quantification of the fatty acids was made with Chrom-Card A/D 1.0 chromatography station (Carlo Erba Instruments) based on heneicosanoic acid as an internal standard.

Acyl-CoA-Esters

Acyl-CoA esters in liver were measured by reversed-phase high-performance liquid chromatography. 100 mg frozen liver was homogenised in ice-cold 1.4 mol/L HClO₄ and 2 mmol/L D-dithiothreitol to obtain 10% (w/v) homogenate, and centrifuged at 12 000×g for 1 min. 122 μl ice-cold 3 mol/L K₂CO₃ with 0.5 mol/L triethanolamnine was added to 500 μl of the supernatant. After 10 min on ice, the solution was centrifuged at 12000×g for 1 min at 4° C. 40 μl of the supernatant was injected onto the high-performance liquid chromatography column, and the acyl-CoA esters were measured according to Demoz et al (39), with the following modifications: elution buffer A was adjusted to pH 5.00, the profile of the gradient elution was as follows: 0 min, 83.5% A; 10 min, 55% A; 17 min, 10% A, and the flow-rate was 1.0 mL/min.

Real-Time Quantitative RT-PCR

Total RNA was purified using Trizol (Gibco BRL), and 1 μg total RNA was reversed-transcribed in a total volume of 100 μl by use of a Reverse transcriptase kit (Applied Biosystems). Reactions in which RNA was omitted served as negative control, and reactions in which RNA was diluted served as standard curves.

Primers and Taqman probe for rat Δ⁹, Δ⁶ and Δ5 desaturases, peroxisome proliferator-activated receptor (PPAR)α and glyceraldehyde-3-phosphate~dehydrogenase (GAPDH) were designed using Primer Express (Applied Biosystems). GAPDH and 18S rRNA were used as endogenous controls. Primers and Taqman probe for 18S rRNA were purchased from Applied Biosystems.

Real-time PCR was carried out in triplicate for each sample on an ABI 7900 sequence detection system (Applied Biosystems). For Δ⁹, Δ^{□} and Δ⁵ desaturases, PPARα and GAPDH, each 20 μl-reaction contained 3 μl first-strand cDNA, 1× Universal Master Mix (Applied Biosystems), 300 nmol/L of each forward and reverse primer, and 250 nmol/L Taqman probe. For 18S rRNA the reaction contained 3 μl first-strand cDNA, 1× Universal Master Mix (Applied Biosystems), and 1×18S probe/primer reaction mix. All reactions were carried out using the following cycle parameters: 50° C. for 2 min and 95° C. for 10 min, followed by 40 cycles of 95° C. for 15 sec and 60° C. for 1 min, as generally recommended by Applied Biosystems. Ct readings (treshold cycle number) for each of the unknown samples were used to calculate the amount of desaturases, PPARα and GAPDH and 18S rRNA. For each sample, results were normalised to GAPDH and 18S rRNA.

The results are reported as means±SEM from 6 animals in each experimental group. Statistical analysis was by one-way Anova Dunett's test (Prism, GraphPad).

### EXAMPLE 1

Preparation of Single-Cell Protein (SCP) Material

A microbial culture comprising Methylococcus capsulatus (Bath), Ralstonia sp., Brevibacillus agri and Aneurinibacillus sp, all commercially available from Norferm Danmark AS, Odense, Denmark is produced in a loop-type fermentor by continuous aerobic fermentation of natural gas in an ammonium/mineral salts medium (AMS) at 45 C, pH 6.5, and at a dilution rate of 0.15 ⁻¹. The AMS medium contains the following per litre: 10 mg NH₃, 75 mg H₃PO₄.2H₂O, 380 mg MgSO₄.7H₂O, 100 mg CaCl₂.2H₂O, 200 mg K₂SO₄, 75 mg FeSO₄.7H₂O, 1.0 mg CuSO₄.5H₂O, 0.96 mg ZnSO₄.7H₂O, 120 μg CoCl₂.6H₂O, 48 μg MnCl₂.4H₂O, 36 μg H₃BO₃, 24 μg NiCl₂.6H₂O and 1.20 μg NaMoO₄.2H₂O.

The fermentor is filled with water which has been heat-sterilized at 125° C. for 10 secs. Addition of the different nutrients is regulated according to their consumption. Continuous fermentation is operated with 2-3% biomass (on a dry weight basis).

A single-cell material having the characteristics given in table 2 is continuously harvested:

TABLE 2
Composition of single-cell protein (SCP) material
Composition (% in product)

| | |
|---|---|
| Crude protein* | 66 |
| Crude fat | 9 |
| Ash | 7 |
| Water | 6 |
| Crude fibre | 1 |
| N-free extract | 11 |
| Total | 100 |

Amino Acids (% in product)

| | |
|---|---|
| Lysine | 4.3 |
| Methionine | 1.9 |
| Cystine | 0.4 |
| Threonine | 3.1 |
| Tryptophan | 1.5 |
| Leucine | 5.2 |
| Isoleucine | 3.2 |
| Valine | 4.2 |
| Tyrosine | 2.8 |
| Phenylalanine | 3.1 |
| Histidine | 1.7 |
| Arginine | 4.1 |
| Alanine | 4.9 |
| Aspartic Acid | 6.2 |
| Glutamic Acid | 7.3 |
| Glycine | 3.4 |
| Proline | 3.0 |
| Serine | 2.5 |
| Total | 62.8 |

Minerals

| Phosphorus | 1.0% | |
|---|---|---|
| Chlorine | 0.7% | |
| Sulphur | 0.5% | |
| Calcium | 0.4% | |
| Potassium | 0.4% | |
| Magnesium | 0.2% | |
| Sodium | 0.1% | |
| Iron | 200 | ppm |
| Copper | 90 | ppm |
| Zinc | 15 | ppm |
| Arsenic | 0.05 | ppm |
| Selenium | 0.02 | ppm |
| Lead | 0.0002 | ppm |
| Cadmium | 0.00002 | ppm |
| Mercury | <0.02 | ppm |

| Vitamins | mg/kg |
|---|---|
| Nicotine acid | 123 |
| Riboflavin B2 | 69 |
| Inositol | 28 |
| Thiamin B1 | 11 |
| Energy | MJ/kg |
| Gross energy | 22.1 |

Other Data

| | |
|---|---|
| Colour | Light brown |
| Flavour | Neutral |
| Particle Size | 100-300 ppm |

*on dry weight basis the crude protein content is approx. 70%.

The biomass is subjected to centrifugation in an industrial continuous centrifuge at 3,000 rpm, followed by ultrafiltration using membranes having an exclusion size of 100,000 Daltons. The resulting product is then subjected to sterilization in a heat exchanger at about 130° C. for about 90 seconds.

### EXAMPLE 2

SCP lowers the Concentration of Plasma Cholesterol

Obese Zucker rats were offered a diet containing 20% SCP as the sole source of protein. The SCP is produced as described in example 1, above.

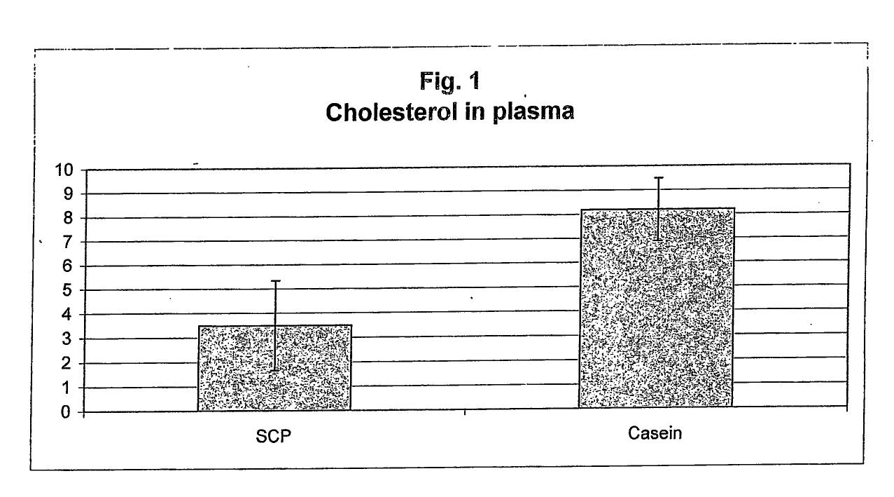

The plasma cholesterol level were reduced by 57% in Zucker rats fed SCP, as compared to rats fed casein as the feed protein. The result is shown in FIG. 1. The result clearly demonstrates that the SCP decreases the levels of cholesterol in the plasma and can be used as a cholesterol lowering agent.

### EXAMPLE 3

SCP Decreases the Concentration of Triacylglycerols in the Liver

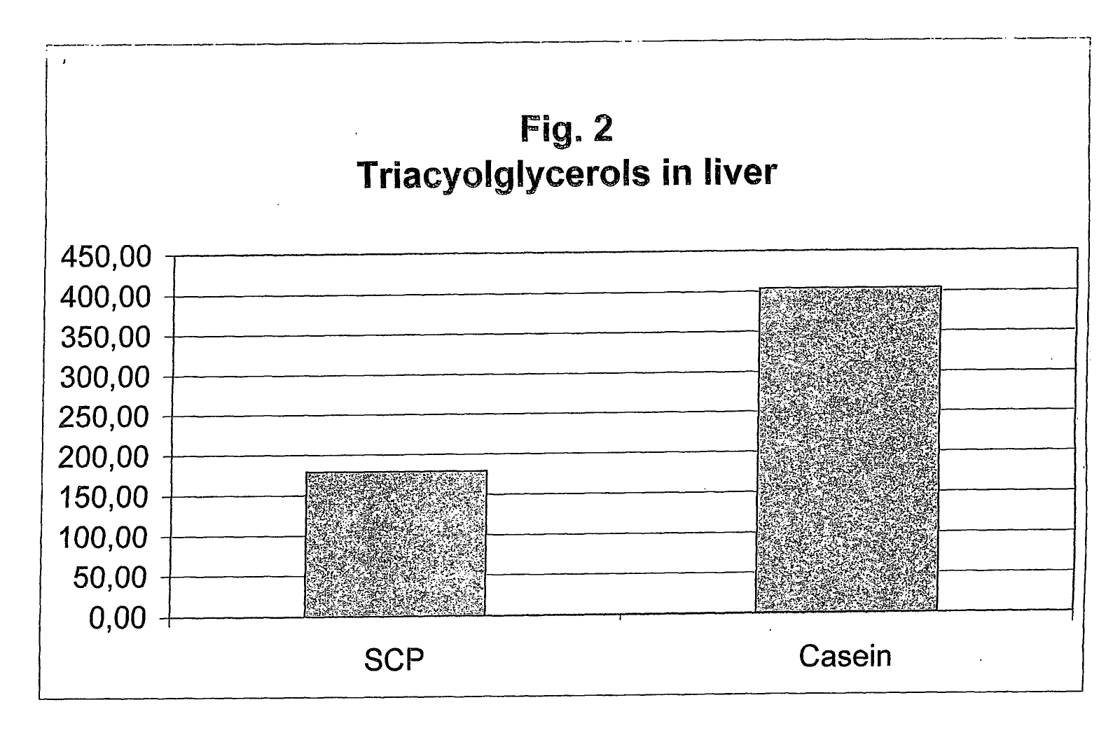

FIG. 2 shows that SCP induces a lowering of the concentration of triacylglycerols (TG) in the liver of about 50%. This indicates that the compound of the present invention can be used as a lipid lowering agent, and for the treatment and prevention of fatty liver.

### EXAMPLE 4

SCP Inhibits the Activity of Acyl-CoA: Cholesterol Acyltransferase (ACAT)

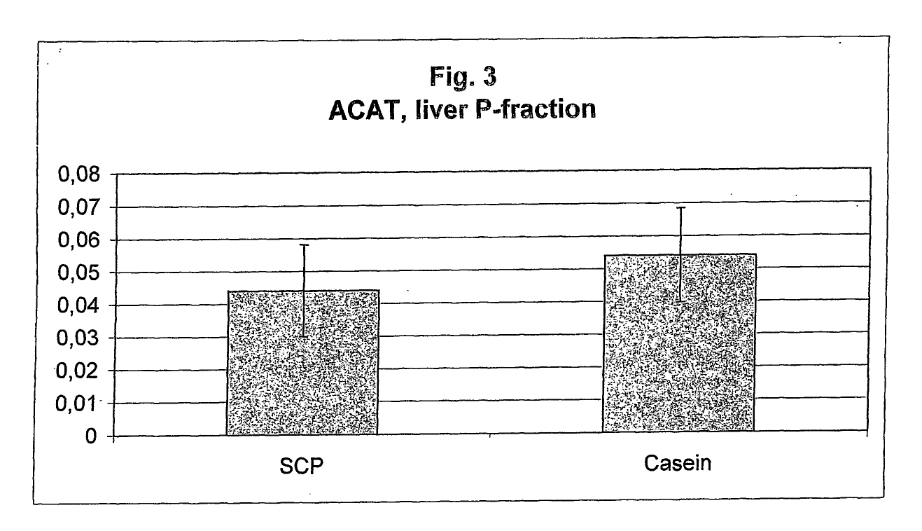

Acyl-CoA:cholesterol acyltransferase (ACAT) catalyses the reaction in which fatty acyl-CoA is esterified to cholesterol. Cholesteryl ester may then be stored in the cytoplasm as lipid droplets or be secreted as part of VLDL together with free cholesterol. Thus, ACAT plays a major role in the VLDL secretion and the subsequent cholesteryl ester accumulation and risk of cardiovascular disease. In the present Zucker rat experiment SCP protein changed the composition of lipid classes in the triacylglycerol-rich lipoprotein fraction, i.e. the cholesteryl ester and phospholipid contents were lower, while the triacylglycerol content was higher than in rats fed casein. FIG. 3 shows that the ACAT activity decreased in rats fed SCP protein as compared to those fed casein. As there is strong evidence that increased ACAT activity plays an important role in the progression of atherosclerosis, this finding indicates that SCP given as a feed supplement or pharmaceutical is cardio protective.

FIG. 3 shows that the ACAT activity was reduced about 20% in rats fed SCP as compared to Zucker rats fed casein.

### EXAMPLE 5

SCP Increases the Mitochondrial β-Oxidation

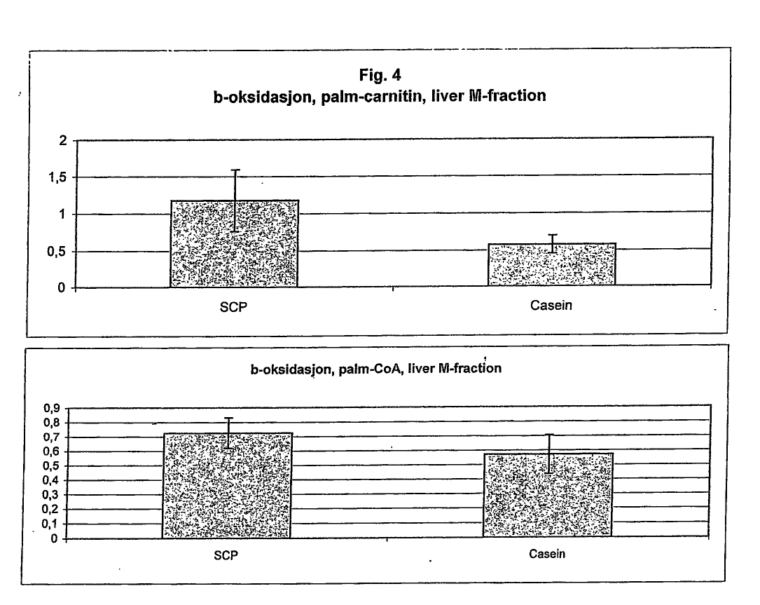

FIG. 4 shows that SCP increases the mitochondrial β-oxidation. Increased fatty acid oxidation is an important factor behind the lipid lowering effect of SCP. The increased fatty acid catabolism will decrease the amount of fatty acids available for esterification, and thereby reduce the production and secretion of VLDL by the liver. From FIG. 4 it can be seen that SCP significantly increased the oxidation of palmitoyl-CoenzymeA compared to control.

### EXAMPLE 6

SCP Interfere with the Lipid Homeostasis

The present data indicate that the SCP material interfere with the lipid homeostasis, and may promote accumulation of endogenous ligands for. In spite of an unchanged hepatic mRNA level of PPARα (data not shown), the fatty acid composition in liver, plasma and triacylglycerol-rich lipoprotein fraction were changed in rats fed SCP as compared to those fed casein, and the changes did not parallel in liver and plasma (Tables 3 and 4). The liver concentrations of the saturated 14:0, 16:0 and 18:0 fatty acids were increased in rats fed SCP, as compared to those fed casein. The liver concentrations of several monounsaturated fatty acids were decreased in rats fed SCP. In contrast to liver, an opposite effect was found in plasma on saturated and monounsaturated fatty acids. In plasma, the saturated fatty acids 14:0 and 16:0 increased by SCP feeding. The monounsaturated fatty acid 18:1n-9 increased about 2 fold in rats fed SCP. A two-fold increase in the 20:4n-6 was seen in the animals fed the SCP. It is therefore anticipated that their hepatic elongase activities were increased. Animals fed SCP showed increased plasma concentrations of 18:2 n-6, while they showed decreased plasma concentrations of 20:4 n-6. As a result their 20:4n-6/18:2n-6 ratio in plasma was reduced. All of the n-3 fatty acids measured in liver were increased in SCP fed rats. 18:3n-3 increased 4 fold in plasma by SCP feeding. 20:5n-3 was significantly increased in SCP-fed rats.

TABLE 3
Fatty acid composition in liver of Zucker
rats fed SCP or casein for 3 weeks¹

| | |
|---|---|
| SCP | Casein |

| | Mean | SD | Mean | SD |
|---|---|---|---|---|
| C14:0 | 1.98 | 0.20 | 1.47 | 0.16 |
| C15:0 | 0.15 | 0.03 | 0.11 | 0.02 |
| C16:0 | 36.84 | 2.59 | 38.77 | 1.90 |
| C17:0 | 0.18 | 0.03 | 0.09 | 0.02 |
| C18:0 | 8.18 | 1.14 | 3.59 | 0.26 |
| C20:0 | 0.05 | 0.01 | 0.03 | 0.00 |
| C22:0 | 0.05 | 0.01 | 0.01 | 0.00 |
| C23:0 | 0.02 | 0.01 | 0.01 | 0.00 |
| C24:0 | 0.11 | 0.02 | 0.03 | 0.01 |
| C14:1n − 5 | 0.12 | 0.00 | 0.12 | 0.02 |
| C16:1n − 9 | 0.68 | 0.06 | 0.72 | 0.08 |
| C16:1n − 7 | 5.28 | 0.14 | 7.73 | 0.85 |
| C17:1n − 8 | 0.12 | 0.02 | 0.14 | 0.02 |
| C18:1n − 9 | 20.29 | 0.96 | 30.33 | 2.40 |
| C18:1n − 7 | 2.22 | 0.38 | 4.16 | 0.18 |
| C20:1n − 11 | 0.02 | 0.01 | 0.01 | 0.00 |
| C20:1n − 9 | 0.06 | 0.02 | 0.08 | 0.01 |
| C20:1n − 7 | 0.02 | 0.01 | 0.03 | 0.01 |
| C24:1n − 9 | 0.02 | 0.01 | 0.01 | 0.00 |
| C20:3n − 9 | 0.03 | 0.01 | 0.04 | 0.01 |
| C18:2n − 6 | 14.21 | 2.52 | 8.73 | 0.85 |
| C20:2n − 6 | 0.12 | 0.04 | 0.04 | 0.01 |
| C18:3n − 6 | 0.57 | 0.12 | 0.35 | 0.06 |
| C20:3n − 6 | 0.66 | 0.15 | 0.11 | 0.01 |
| C20:4n − 6 | 4.69 | 1.00 | 2.03 | 0.36 |
| C22:4n − 6 | 0.22 | 0.06 | 0.12 | 0.04 |
| C22:5n − 6 | 0.15 | 0.04 | 0.08 | 0.02 |
| C18:3n − 3 | 0.76 | 0.16 | 0.30 | 0.03 |
| C18:4n − 3 | 0.08 | 0.02 | 0.02 | 0.00 |
| C20:4n − 3 | 0.06 | 0.03 | 0.01 | 0.00 |
| C20:5n − 3 | 0.23 | 0.06 | 0.04 | 0.01 |
| C22:5n − 3 | 0.59 | 0.16 | 0.15 | 0.04 |
| C22:6n − 3 | 1.23 | 0.36 | 0.53 | 0.11 |

TABLE 4
Fatty acid composition in plasma of Zucker
rats fed SCP or casein for 3 weeks¹

| | |
|---|---|
| SCP | Casein |

| | Mean | SD | Mean | SD |
|---|---|---|---|---|
| C12:0 | 0.05 | 0.01 | 0.02 | 0.01 |
| C14:0 | 0.99 | 0.15 | 0.31 | 0.03 |
| C15:0 | 0.13 | 0.02 | 0.08 | 0.01 |
| C16:0 | 24.59 | 2.08 | 17.48 | 0.97 |
| C17:0 | 0.14 | 0.01 | 0.09 | 0.01 |
| C18:0 | 10.95 | 1.30 | 12.62 | 0.71 |
| C20:0 | 0.09 | 0.02 | 0.06 | 0.01 |
| C22:0 | 0.19 | 0.07 | 0.21 | 0.03 |
| C23:0 | 0.07 | 0.02 | 0.09 | 0.01 |
| C24:0 | 0.30 | 0.10 | 0.31 | 0.05 |
| C14:1n − 5 | 0.06 | 0.02 | 0.01 | 0.01 |
| C16:1n − 9 | 0.40 | 0.06 | 0.27 | 0.06 |
| C16:1n − 7 | 2.70 | 2.04 | 1.13 | 0.89 |
| C17:1n − 8 | 0.07 | 0.02 | 0.05 | 0.01 |
| C18:1n − 9 | 11.54 | 2.38 | 6.39 | 1.15 |
| C18:1n − 7 | 1.43 | 0.21 | 1.47 | 0.11 |
| C20:1n − 9 | 0.07 | 0.02 | 0.05 | 0.01 |
| C20:1n − 7 | 0.04 | 0.01 | 0.03 | 0.00 |
| C24:1n − 9 | 0.14 | 0.06 | 0.25 | 0.03 |
| C20:3n − 9 | 0.05 | 0.01 | 0.08 | 0.02 |
| C18:2n − 6 | 22.99 | 2.14 | 11.92 | 1.22 |
| C18:3n − 6 | 0.60 | 0.09 | 0.40 | 0.10 |
| C20:2n − 6 | 0.14 | 0.02 | 0.08 | 0.01 |
| C20:3n − 6 | 1.29 | 0.12 | 0.47 | 0.07 |
| C20:4n − 6 | 16.14 | 3.76 | 41.56 | 1.90 |
| C22:4n − 6 | 0.34 | 0.07 | 0.35 | 0.05 |
| C22:5n − 6 | 0.22 | 0.03 | 0.39 | 0.05 |
| C18:3n − 3 | 0.92 | 0.17 | 0.22 | 0.05 |
| C18:4n − 3 | 0.05 | 0.03 | 0.00 | 0.00 |
| C20:4n − 3 | 0.09 | 0.03 | 0.01 | 0.01 |
| C20:5n − 3 | 0.66 | 0.09 | 0.24 | 0.06 |
| C22:5n − 3 | 0.89 | 0.11 | 0.52 | 0.08 |
| C22:6n − 3 | 1.68 | 0.33 | 2.85 | 0.24 |

### EXAMPLE 7

SCP Lowers the Concentration of Homocystein in Plasma

Increased levels of homocysteine, i.e. hyperhomocysteinemia has been proposed to be associated with arterial diseases, and we thus measured the levels of homocysteine in the plasma samples from rats.

Total plasma homocysteine was measured by a fully automated fluorescence assay. 30 μL plasma was reduced by 30 μl NaBH4/DMSO solution (6 mol/L). After 1.5 min 20 μl of the fluorescence reagent monobromobimane (25 mmol/L) in acetonitrile was added and allowed to react for 3 min. 20 μl of the sample was then immediately analysed with HPLC by injection on a strong cation-exchange column, and then by column switching into a cyclohexyl silica column. The SCX column was eluted isocratically and the CH column was eluted with a linear methanol gradient (17-35% in 5 min) in 20 mmol/L formate buffer. The homocysteine was eluted at a retention time of 4.5 min. The results are given in table 5.

TABLE 5
Plasma concentration of homocysteine

| | Plasma concentration (μmol/L) |
|---|---|
| Control (casein) | 1.37 ± 0.27 |
| SCP | 1.09 ± 0.18 |

1-21. (canceled)

22. A method of treating or preventing a disease comprising administering to an animal in need of such treatment, a pharmaceutical or nutritional composition comprising a single cell protein material.

23. The method of claim 22, wherein the disease is atherosclerosis, coronary heart disease, stenosis, thrombosis, myocardial infarction, stroke or fatty liver.

24. The method of claim 22, wherein the disease is hypercholestrolemia.

25. The method of claim 22, wherein the disease is hyperhomocysteinemia.

26. A cardio protective pharmaceutical or nutritional composition comprising a single cell protein material.

27. A method of changing the fatty acyl profile and for improving the lipid homeostasis of an animal comprising administering to an animal in need of such treatment, a pharmaceutical or nutritional composition comprising a single cell protein material.

28. The method of any one of claims 22 or 27, wherein said animal is a human.

29. The method of any one of claims 22 or 27, wherein said animal is an agricultural animal selected from the group consisting of gallinaceous birds, bovine, ovine, caprine and porcine.

30. The method of any one of claims 22 or 27, wherein said animal is a domestic animal.

31. The method of any one of claims 22 or 27, wherein said animal is a fish or shellfish.

32. The method of any one of claims 22 or 27, wherein said single-cell protein material is derived from a microbial culture comprising methanotrophic bacteria.

33. The method of claim 32, wherein said microbial culture further comprises one or more species of heterotrophic bacteria.

34. The method of claim 32, wherein said microbial culture comprises a combination of microbes selected from the group consisting of Methylococcus capsulatus, Ralstonia sp., Brevibacillus agri and Aneurinibacillus sp.

35. The method of claim 32, wherein the methanotrophic bacteria is Methyloccus capsulatus.

36. The method of claim 32, wherein the microbial culture is produced by continuous fermnentation, preferably operated with 2-3% biomass (on a dry weight basis).

37. The method of claim 32, wherein the microbial culture after fermentation is subjected to centrifugation in an industrial continuous centrifuge, preferably at 3,000 rpm, followed by ultrafiltration using membranes having an exclusion size of preferable 100,000 Daltons to produce the single cell protein material.

38. The method of claim 37, wherein the single-cell protein material is further subjected to a sterilization step, preferable in a heat exchanger.

39. The method of claim 37, wherein the single-cell protein material is further subjected to a homogenization step.

40. The method of claim 32, wherein the single-cell protein material is dried by spray drying.

41. The method of claim 40, wherein prior to spray drying the single cell protein material is held in a storage tank at a temperature of less than 20° C. and a pH of less than about 6.5.

42. The method of claim 32, wherein said microbial culture is a fermentation on hydrocarbon fractions or a natural gas.

43. The nutritional composition of any one of claims 22 or 27, wherein the composition is a food grade product or additive.
